(19) United States
(12) Patent Application Publication
(10) Pub. No.: US 2013/0315975 A1
(43) Pub. Date: Nov. 28, 2013

(54) USE OF KERATINOCYTES AS A BIOLOGICALLY ACTIVE SUBSTANCE IN THE TREATMENT OF WOUNDS, SUCH AS DIABETIC WOUNDS, OPTIONALLY IN COMBINATION WITH A DPP-4 INHIBITOR

(71) Applicants: Thomas KLEIN, Radolfzell (DE); Michael MARK, Biberach an der Riss (DE)

(72) Inventors: Thomas KLEIN, Radolfzell (DE); Michael MARK, Biberach an der Riss (DE)

(73) Assignee: BOEHRINGER INGELHEIM INTERNATIONAL GMBH, Ingelheim am Rhein (DE)

(21) Appl. No.: 13/900,750

(22) Filed: May 23, 2013

(30) Foreign Application Priority Data
May 25, 2012 (EP) 12004077.9

(51) Int. Cl. A61K 35/36; A61K 47/48; A61K 9/70; A61K 45/06; A61K 31/522

(52) U.S. Cl. CPC A61K 35/36; A61K 45/06; A61K 31/522; A61K 9/7007; A61K 47/48976
USPC 424/443; 424/93.7; 435/374

(57) ABSTRACT

The invention relates to new keratinocytes which may be cultured in vitro and the use thereof for preparing a product which can be used to treat acute and chronic wounds, in combination with a DPP-4 inhibitor.

### FIELD OF THE INVENTION

The invention relates to keratinocytes which may be cultivated in vitro for use to treat acute or chronic wounds, particularly diabetic wounds (e.g. diabetic foot or ulcus), optionally in combination with a DPP-4 inhibitor, particularly linagliptin.

### SUMMARY OF THE INVENTION

The invention relates to keratinocytes which may be cultivated in vitro for use to treat acute or chronic wounds, particularly diabetic wounds (e.g. diabetic foot or ulcus), optionally in combination with a DPP-4 inhibitor, particularly linagliptin.

Further, the invention contemplates these keratinocytes in combination with a DPP-4 inhibitor, particularly linagliptin, e.g. for use in preparing a pharmaceutical composition or medical product (e.g. a biologically active wound dressing, BAWD), which can be used to treat acute or chronic wounds, particularly diabetic wounds (e.g. diabetic foot or ulcus).

Further, the invention relates to a pharmaceutical composition or product (e.g. a pharmaceutical composition for topical use or a biologically active wound dressing, BAWD) containing such keratinocytes and a DPP-4 inhibitor, particularly linagliptin, and optionally one or more pharmaceutically acceptable carriers.

Further, the invention relates to a method of treating acute or chronic wounds, particularly diabetic wounds (e.g. diabetic foot or ulcus), comprising administering or applying an effective amount of such keratinocytes and a DPP-4 inhibitor, particularly linagliptin, to the patient in need thereof (e.g. by simultaneous topical application of such active components, such as e.g. in the same topical application form).

The invention was thus made in the field of medicine, specifically in the field of wound healing by tissue engineering.

### DETAILED DESCRIPTION OF THE INVENTION

Within the scope of the present invention it has now been found that keratinocytes, optionally in combination with a DPP-4 inhibitor, preferably linagliptin, each as defined herein, have properties, which make them suitable for the purpose of this invention.

Linagliptin and the keratinocytes (e.g. in form of a BAWD) of this invention hold some inherent characteristics that are particularly suitable for treating wounds (particularly diabetic wounds), particularly in combination. For example, linagliptin attenuates inflammation and accelerates epithelialization (wound closure, re-epithelialization) of diabetic wounds. For further example, the keratinocytes of this invention (particularly BAWD of the human keratinocytes (KC-BI-1) and hyaluronic acid matrix as carrier) drives and increases the formation of functional new tissue from chronically-disturbed wounds (such as e.g. by releasing growth factors), where the new tissue is vascularized (characterized by high numbers of new blood vessels), and improves wound healing (re-epithelialization) despite the presence of a diabetic phenotype and massive hyperglycemia.

Thus, the present invention provides a pharmaceutical combination or composition comprising or consisting essentially of keratinocytes (particularly human keratinocytes, particularly KC-BI-1, e.g. in the form of a BAWD) and a DPP-4 inhibitor (particularly linagliptin), each as described herein, such as e.g. for simultaneous, sequential or separate use in therapy.

The present invention further provides a method for treating wounds (particularly diabetic wounds) in a patient (particularly human patient), said method comprising administering or applying an effective amount of keratinocytes (particularly human keratinocytes, particularly KC-BI-1, e.g. in the form of a BAWD) and a DPP-4 inhibitor (particularly linagliptin), each as described herein, particularly in combination including in alternation, to the patient.

Within this invention it is to be understood that the combinations or combined uses according to this invention may envisage the simultaneous, sequential or separate administration of the active components or ingredients.

In this context, “combination” or “combined” within the meaning of this invention may include, without being limited, fixed and non-fixed (e.g. free) forms (including kits) and uses, such as e.g. the simultaneous, sequential or separate use of the components or ingredients.

The combined administration of this invention may take place by administering the active components together, such as e.g. by administering them simultaneously in one single or in two separate formulations or dosage forms. Alternatively, the administration may take place by administering the active components sequentially, such as e.g. successively in two separate formulations or dosage forms.

For the combination therapy of this invention the active components may be administered separately (which implies that they are formulated separately) or formulated altogether (which implies that they are formulated in the same preparation or in the same dosage form). Hence, the administration of one element of the combination of the present invention may be prior to, concurrent to, or subsequent to the administration of the other element of the combination.

A DPP-4 inhibitor within the meaning of the present invention includes, without being limited to, any of those DPP-4 inhibitors mentioned hereinabove and hereinbelow, preferably orally and/or subcutaneously and/or topically active DPP-4 inhibitors.

In a first embodiment (embodiment A), a DPP-4 inhibitor in the context of the present invention is any DPP-4 inhibitor of

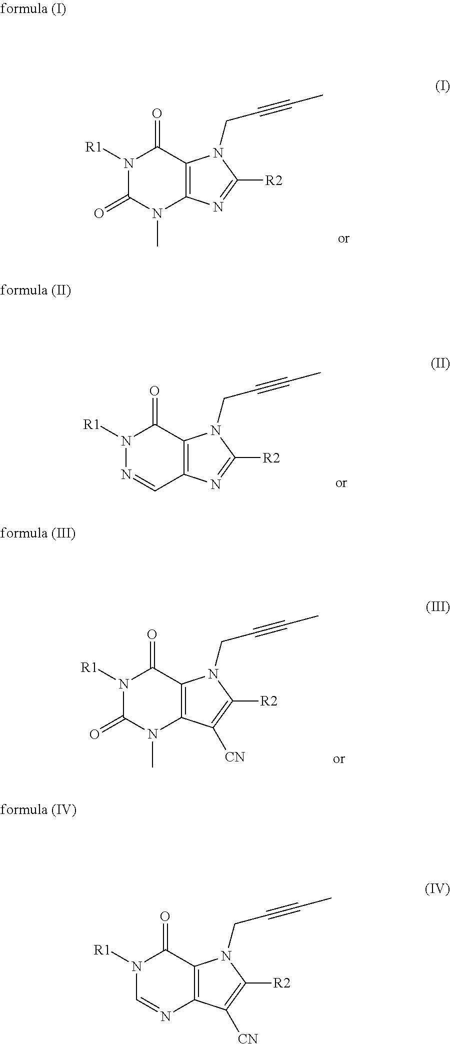

wherein R1 denotes ([1,5]naphthyridin-2-yl)methyl, (quinazolin-2-yl)methyl, (quinoxalin-6-yl)methyl, (4-methyl-quinazolin-2-yl)methyl, 2-cyano-benzyl, (3-cyano-quinolin-2-yl)methyl, (3-cyano-pyridin-2-yl)methyl, (4-methyl-pyrimidin-2-yl)methyl, or (4,6-dimethyl-pyrimidin-2-yl)methyl and R2 denotes 3-(R)-amino-piperidin-1-yl, (2-amino-2-methyl-propyl)-methylamino or (2-(S)-amino-propyl)-methylamino,
or its pharmaceutically acceptable salt.

Regarding the first embodiment (embodiment A), preferred DPP-4 inhibitors are any or all of the following compounds and their pharmaceutically acceptable salts:
1-[(4-methyl-quinazolin-2-yl)methyl]-3-methyl-7-(2-butyn-1-yl)-8-(3-(R)-amino-piperidin-1-yl)-xanthine (compare WO 2004/018468, example 2(142)):

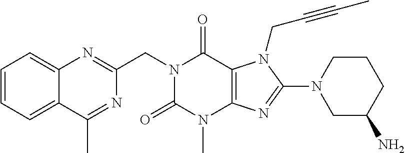 [SMILES: CC#CCn1c(N2CCC[C@@H](N)C2)nc2c1c(=O)n(Cc1nc(C)c3ccccc3n1)c(=O)n2C]

1-[([1,5]naphthyridin-2-yl)methyl]-3-methyl-7-(2-butyn-1-yl)-8-((R)-3-amino-piperidin-1-yl)-xanthine (compare WO 2004/018468, example 2(252)):

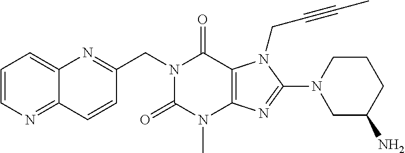 [SMILES: CC#CCn1c(N2CCC[C@@H](N)C2)nc2c1c(=O)n(Cc1ccc3ncccc3n1)c(=O)n2C]

1-[(Quinazolin-2-yl)methyl]-3-methyl-7-(2-butyn-1-yl)-8-((R)-3-amino-piperidin-1-yl)-xanthine (compare WO 2004/018468, example 2(80)):

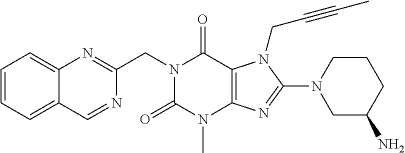 [SMILES: CC#CCn1c(N2CCC[C@@H](N)C2)nc2c1c(=O)n(Cc1ncc3ccccc3n1)c(=O)n2C]

2-((R)-3-Amino-piperidin-1-yl)-3-(but-2-yinyl)-5-(4-methyl-quinazolin-2-ylmethyl)-3,5-dihydro-imidazo[4,5-d]pyridazin-4-one (compare WO 2004/050658, example 136):

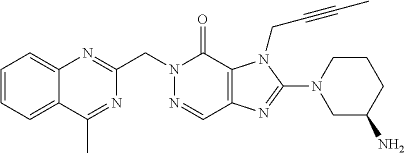 [SMILES: CC#CCn1c(N2CCC[C@@H](N)C2)nc2cnn(Cc3nc(C)c4ccccc4n3)c(=O)c21]

1-[(4-Methyl-quinazolin-2-yl)methyl]-3-methyl-7-(2-butyin-1-yl)-8-[(2-amino-2-methyl-propyl)-methylamino]-xanthine (compare WO 2006/029769, example 2(1)):

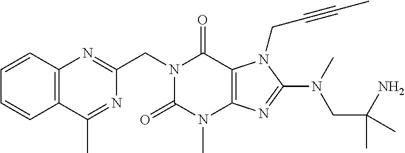 [SMILES: CC#CCn1c(N(C)CC(C)(C)N)nc2c1c(=O)n(Cc1nc(C)c3ccccc3n1)c(=O)n2C]

1-[(3-Cyano-quinolin-2-yl)methyl]-3-methyl-7-(2-butyn-1-yl)-8-((R)-3-amino-piperidin-1-yl)-xanthine (compare WO 2005/085246, example 1(30)):

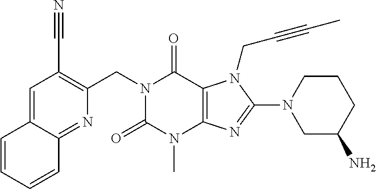 [SMILES: CC#CCn1c(N2CCC[C@@H](N)C2)nc2c1c(=O)n(Cc1nc3ccccc3cc1C#N)c(=O)n2C]

1-(2-Cyano-benzyl)-3-methyl-7-(2-butyn-1-yl)-8-((R)-3-amino-piperidin-1-yl)-xanthine (compare WO 2005/085246, example 1(39)):

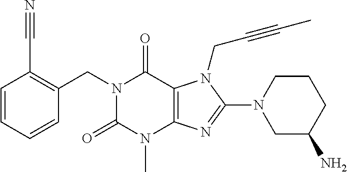 [SMILES: CC#CCn1c(N2CCC[C@@H](N)C2)nc2c1c(=O)n(Cc1ccccc1C#N)c(=O)n2C]

1-[(4-Methyl-quinazolin-2-yl)methyl]-3-methyl-7-(2-butyn-1-yl)-8-[(S)-(2-amino-propyl)-methylamino]-xanthine (compare WO 2006/029769, example 2(4)):

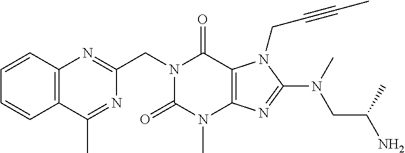 [SMILES: CC#CCn1c(N(C)C[C@H](C)N)nc2c1c(=O)n(Cc1nc(C)c3ccccc3n1)c(=O)n2C]

1-[(3-Cyano-pyridin-2-yl)methyl]-3-methyl-7-(2-butyn-1-yl)-8-((R)-3-amino-piperidin-1-yl)-xanthine (compare WO 2005/085246, example 1(52)):

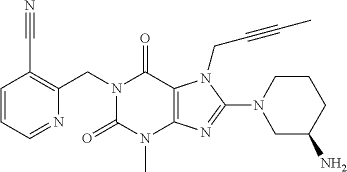 [SMILES: CC#CCn1c(N2CCC[C@@H](N)C2)nc2c1c(=O)n(Cc1ncccc1C#N)c(=O)n2C]

1-[(4-Methyl-pyrimidin-2-yl)methyl]-3-methyl-7-(2-butyn-1-yl)-8-((R)-3-amino-piperidin-1-yl)-xanthine (compare WO 2005/085246, example 1(81)):

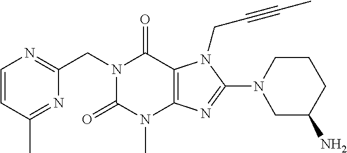 [SMILES: CC#CCn1c(N2CCC[C@@H](N)C2)nc2c1c(=O)n(Cc1nccc(C)n1)c(=O)n2C]

1-[(4,6-Dimethyl-pyrimidin-2-yl)methyl]-3-methyl-7-(2-butyn-1-yl)-8-((R)-3-amino-piperidin-1-yl)-xanthine (compare WO 2005/085246, example 1(82)):

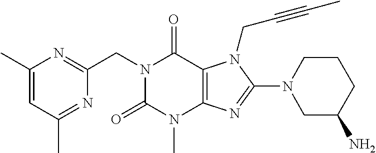 [SMILES: CC#CCn1c(N2CCC[C@@H](N)C2)nc2c1c(=O)n(Cc1nc(C)cc(C)n1)c(=O)n2C]

1-[(Quinoxalin-6-yl)methyl]-3-methyl-7-(2-butyn-1-yl)-8-((R)-3-amino-piperidin-1-yl)-xanthine (compare WO 2005/085246, example 1(83)):

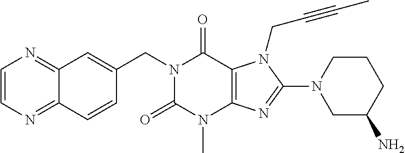 [SMILES: CC#CCn1c(N2CCC[C@@H](N)C2)nc2c1c(=O)n(Cc1ccc3nccnc3c1)c(=O)n2C]

These DPP-4 inhibitors are distinguished from structurally comparable DPP-4 inhibitors, as they combine exceptional potency and a long-lasting effect with favourable pharmacological properties, receptor selectivity and a favourable side-effect profile or bring about unexpected therapeutic advantages or improvements when combined with other pharmaceutical active substances. Their preparation is disclosed in the publications mentioned.

In a second embodiment (embodiment B), a DPP-4 inhibitor in the context of the present invention is a DPP-4 inhibitor selected from the group consisting of
sitagliptin, vildagliptin, saxagliptin, alogliptin, gemigliptin,
(2S)-1-{[2-(5-Methyl-2-phenyl-oxazol-4-yl)-ethylamino]-acetyl}-pyrrolidine-2-carbonitrile,
(2S)-1-{[1,1,-Dimethyl-3-(4-pyridin-3-yl-imidazol-1-yl)-propylamino]-acetyl}-pyrrolidine-2-carbonitrile,
(S)-1-((2S,3S,11bS)-2-Amino-9,10-dimethoxy-1,3,4,6,7,11b-hexahydro-2H-pyrido[2,1-a]isoquinolin-3-yl)-4-fluoromethyl-pyrrolidin-2-one,
(3,3-Difluoropyrrolidin-1-yl)-((2S,4S)-4-(4-(pyrimidin-2-yl)piperazin-1-yl)pyrrolidin-2-yl)methanone,
(1((3S,4S)-4-amino-1-(4-(3,3-difluoropyrrolidin-1-yl)-1,3,5-triazin-2-yl)pyrrolidin-3-yl)-5,5-difluoropiperidin-2-one,
(2S,4S)-1-{2-[(3S,1R)-3-(1H-1,2,4-Triazol-1-ylmethyl)cyclopentylamino]-acetyl}-4-fluoropyrrolidine-2-carbonitrile,
(R)-2-[6-(3-Amino-piperidin-1-yl)-3-methyl-2,4-dioxo-3,4-dihydro-2H-pyrimidin-1-ylmethyl]-4-fluoro-benzonitrile,
5-{(S)-2-[2-((S)-2-Cyano-pyrrolidin-1-yl)-2-oxo-ethylamino]-propyl}-5-(1H-tetrazol-5-yl)-10,11-dihydro-5H-dibenzo[a,d]cycloheptene-2,8-dicarboxylic acid bis-dimethylamide,
3-{(2S,4S)-4-[4-(3-Methyl-1-phenyl-1H-pyrazol-5-yl)piperazin-1-yl]pyrrolidin-2-ylcarbonyl}thiazolidine,
[(2R)-1-{[(3R)-pyrrolidin-3-ylamino]acetyl}pyrrolidin-2-yl]boronic acid,
(2S,4S)-1-[2-[(4-ethoxycarbonylbicyclo[2.2.2]oct-1-yl)amino]acetyl]-4-fluoropyrrolidine-2-carbonitrile,
2-({6-[(3R)-3-amino-3-methylpiperidin-1-yl]-1,3-dimethyl-2,4-dioxo-1,2,3,4-tetrahydro-5H-pyrrolo[3,2-d]pyrimidin-5-yl}methyl)-4-fluorobenzonitrile,
6-[(3R)-3-amino-piperidin-1-yl]-5-(2-chloro-5-fluoro-benzyl)-1,3-dimethyl-1,5-dihydro-pyrrolo[3,2-d]pyrimidine-2,4-dione, and
(S)-2-methylpyrazolo[1,5-a]primidine-6-carboxylic acid {2-[(2-cyanopyrrolidin-1-yl)-2-oxoethylamino]-2-methylpropyl}amide,
   or its pharmaceutically acceptable salt.

A more preferred DPP-4 inhibitor among the abovementioned DPP-4 inhibitors of embodiment A of this invention is 1-[(4-methyl-quinazolin-2-yl)methyl]-3-methyl-7-(2-butyn-1-yl)-8-(3-(R)-amino-piperidin-1-yl)-xanthine, particularly the free base thereof (which is also known as linagliptin or BI 1356).

Preferably the DPP-4 inhibitor of this invention is selected from the group consisting of linagliptin, sitagliptin, vildagliptin, alogliptin, saxagliptin, teneligliptin, anagliptin, gemigliptin and dutogliptin, or a pharmaceutically acceptable salt of one of the herein mentioned DPP-4 inhibitors, or a prodrug thereof.

A particularly preferred DPP-4 inhibitor to be emphasized within the present invention is linagliptin. The term “linagliptin” as employed herein refers to linagliptin or a pharmaceutically acceptable salt thereof, including hydrates and solvates thereof, and crystalline forms thereof, preferably linagliptin refers to 1-[(4-methyl-quinazolin-2-yl)methyl]-3-methyl-7-(2-butyn-1-yl)-8-(3-(R)-amino-piperidin-1-yl)-xanthine. Crystalline forms are described in WO 2007/128721. Methods for the manufacture of linagliptin are described in the patent applications WO 2004/018468 and WO 2006/048427 for example. Linagliptin is distinguished from structurally comparable DPP-4 inhibitors, as it combines exceptional potency and a long-lasting effect with favourable pharmacological properties, receptor selectivity and a favourable side-effect profile or bring about unexpected therapeutic advantages or improvements in therapy.

With respect to embodiment A, the methods of synthesis for the DPP-4 inhibitors according to embodiment A of this invention are known to the skilled person. Advantageously, the DPP-4 inhibitors according to embodiment A of this invention can be prepared using synthetic methods as described in the literature. Thus, for example, purine derivatives of formula (I) can be obtained as described in WO 2002/068420, WO 2004/018468, WO 2005/085246, WO 2006/029769 or WO 2006/048427, the disclosures of which are incorporated herein.

Purine derivatives of formula (II) can be obtained as described, for example, in WO 2004/050658 or WO 2005/110999, the disclosures of which are incorporated herein.

Purine derivatives of formula (III) and (IV) can be obtained as described, for example, in WO 2006/068163, WO 2007/071738 or WO 2008/017670, the disclosures of which are incorporated herein. The preparation of those DPP-4 inhibitors, which are specifically mentioned hereinabove, is disclosed in the publications mentioned in connection therewith. Polymorphous crystal modifications and formulations of particular DPP-4 inhibitors are disclosed in WO 2007/128721 and WO 2007/128724, respectively, the disclosures of which are incorporated herein in their entireties. Formulations of particular DPP-4 inhibitors with metformin or other combination partners are described in WO 2009/121945, the disclosure of which is incorporated herein in its entirety.

With respect to embodiment B, the methods of synthesis for the DPP-4 inhibitors of embodiment B are described in the scientific literature and/or in published patent documents, particularly in those cited herein.

In an embodiment, the DPP-4 inhibitor according to the invention is preferably administered orally.

Suitable doses and dosage forms of the DPP-4 inhibitors may be determined by a person skilled in the art and may include those described herein or in the relevant references.

For pharmaceutical application in warm-blooded vertebrates, particularly humans, the compounds of this invention are usually used in dosages from 0.001 to 100 mg/kg body weight, preferably at 0.01-15 mg/kg or 0.1-15 mg/kg, in each case 1 to 4 times a day. For this purpose, the compounds, optionally combined with other active substances, may be incorporated together with one or more inert conventional carriers and/or diluents, e.g. with corn starch, lactose, glucose, microcrystalline cellulose, magnesium stearate, polyvinylpyrrolidone, citric acid, tartaric acid, water, water/ethanol, water/glycerol, water/sorbitol, water/polyethylene glycol, propylene glycol, cetylstearyl alcohol, carboxymethylcellulose or fatty substances such as hard fat or suitable mixtures thereof into conventional galenic preparations such as plain or coated tablets, capsules, powders, suspensions or suppositories.

The pharmaceutical compositions according to this invention comprising the DPP-4 inhibitors as defined herein are thus prepared by the skilled person using pharmaceutically acceptable formulation excipients as described in the art and appropriate for the desired route of administration. Examples of such excipients include, without being restricted to diluents, binders, carriers, fillers, lubricants, flow promoters, crystallisation retardants, disintegrants, solubilizers, colorants, pH regulators, surfactants and emulsifiers.

Oral formulations or dosage forms of the DPP-4 inhibitor of this invention may be prepared according to known techniques.

A pharmaceutical composition or dosage form (e.g. oral tablet) of a DPP-4 inhibitor according to embodiment A of the invention may typically contain as excipients (in addition to an active ingredient), for example: one or more diluents, a binder, a disintegrant, and a lubricant, preferably each as disclosed herein-below. In an embodiment, the disintegrant may be optional.

Examples of suitable diluents for compounds according to embodiment A include cellulose powder, calcium hydrogen phosphate, erythritol, low substituted hydroxypropyl cellulose, mannitol, pregelatinized starch or xylitol.

Examples of suitable lubricants for compounds according to embodiment A include talc, polyethyleneglycol, calcium behenate, calcium stearate, hydrogenated castor oil or magnesium stearate.

Examples of suitable binders for compounds according to embodiment A include copovidone (copolymerisates of vinylpyrrolidon with other vinylderivates), hydroxypropyl methylcellulose (HPMC), hydroxypropylcellulose (HPC), polyvinylpyrrolidon (povidone), pregelatinized starch, or low-substituted hydroxypropylcellulose (L-HPC).

Examples of suitable disintegrants for compounds according to embodiment A include corn starch or crospovidone.

Suitable methods of preparing (oral) preparations or dosage forms of the DPP-4 inhibitors according to embodiment A of the invention are:

direct tabletting of the active substance in powder mixtures with suitable tabletting excipients;
granulation with suitable excipients and subsequent mixing with suitable excipients and subsequent tabletting as well as film coating; or
packing of powder mixtures or granules into capsules.

Suitable granulation methods are:

wet granulation in the intensive mixer followed by fluidised bed drying; one-pot granulation;
fluidised bed granulation; or
dry granulation (e.g. by roller compaction) with suitable excipients and subsequent tabletting or packing into capsules.

An exemplary composition for oral use (e.g. tablet core) of a DPP-4 inhibitor according to embodiment A of the invention comprises the first diluent mannitol, pregelatinized starch as a second diluent with additional binder properties, the binder copovidone, the disintegrant corn starch, and magnesium stearate as lubricant; wherein copovidone and/or corn starch may be optional.

A tablet of a DPP-4 inhibitor according to embodiment A of the invention may be film coated, preferably the film coat comprises hydroxypropylmethylcellulose (HPMC), polyethylene glycol (PEG), talc, titanium dioxide and iron oxide (e.g. red and/or yellow).

In another embodiment, a DPP-4 inhibitor of the invention may be for topic use and thus e.g. in the form of an ointment. Such a topical preparation typically comprises the active ingredient(s) with suitable carrier materials for topical preparations, such as, for example, glycerides, semi-synthetic and synthetic glycerides, hydrogenated oils, liquid waxes, liquid paraffins, liquid fatty alcohols, sterols, polyethylene glycols and/or cellulose derivatives.

For details on dosage forms, formulations and administration of DPP-4 inhibitors of this invention, reference is made to scientific literature and/or published patent documents, particularly to those cited herein.

In a further embodiment, the DPP-4 inhibitor according to the invention may be administered topically. In another embodiment, the keratinocytes are preferably administered topically as well. A topical preparation (e.g. in form of an ointment, cream, lotion, paste, gel, dressing, patch) of the DPP-4 inhibitor, optionally in combination with the keratinocytes, typically comprises the active ingredient(s) with suitable carrier materials for topical preparations.

Examples of carrier materials of topical cream or ointment-type preparations typically include (without being limited to), emollients or softening agents, emulsifying or thickening agents, humectants and/or moisturisers, gelling agents, preservatives, oils, waxes, solvents, fragrances, dyes, antioxidants, antifoaming agents, stabilising agents, pH adjusters and the like, such as e.g. glycerides, semi-synthetic and synthetic glycerides, hydrogenated oils, liquid waxes, liquid paraffins, liquid fatty alcohols, sterols, polyethylene glycols and/or cellulose derivatives.

Examples of carrier materials of dressings or patches typically include (without being limited to) polymeric matrix materials, which may be biocompatible synthetic or natural materials.

With respect to the first embodiment (embodiment A), the dosage typically required of the DPP-4 inhibitors mentioned herein in embodiment A when administered intravenously is 0.1 mg to 10 mg, preferably 0.25 mg to 5 mg, and when administered orally is 0.5 mg to 100 mg, preferably 2.5 mg to 50 mg or 0.5 mg to 10 mg, more preferably 2.5 mg to 10 mg or 1 mg to 5 mg, in each case 1 to 4 times a day. Thus, e.g. the dosage of 1-[(4-methyl-quinazolin-2-yl)methyl]-3-methyl-7-(2-butyn-1-yl)-8-(3-(R)-amino-piperidin-1-yl)-xanthine when administered orally is 0.5 mg to 10 mg per patient per day, preferably 2.5 mg to 10 mg or 1 mg to 5 mg per patient per day.

A dosage form prepared with a pharmaceutical composition comprising a DPP-4 inhibitor mentioned herein in embodiment A contain the active ingredient in a dosage range of 0.1-100 mg. Thus, e.g. particular oral dosage strengths of 1-[(4-methyl-quinazolin-2-yl)methyl]-3-methyl-7-(2-butyn-1-yl)-8-(3-(R)-amino-piperidin-1-yl)-xanthine are 0.5 mg, 1 mg, 2.5 mg, 5 mg and 10 mg.

A special embodiment of the DPP-4 inhibitors of this invention refers to those orally administered DPP-4 inhibitors which are therapeutically efficacious at low dose levels, e.g. at oral dose levels <100 mg or <70 mg per patient per day, preferably <50 mg, more preferably <30 mg or <20 mg, even more preferably from 1 mg to 10 mg, particularly from 1 mg to 5 mg (more particularly 5 mg), per patient per day (if required, divided into 1 to 4 single doses, particularly 1 or 2 single doses, which may be of the same size, preferentially, administered orally once- or twice daily (more preferentially once-daily), advantageously, administered at any time of day, with or without food. Thus, for example, the daily oral amount 5 mg BI 1356 can be given in an once daily dosing regimen (i.e. 5 mg BI 1356 once daily) or in a twice daily dosing regimen (i.e. 2.5 mg BI 1356 twice daily), at any time of day, with or without food.

The dosage of the active components in the combinations or compositions in accordance with the present invention may be varied, although the amount of the active ingredients shall be such that a suitable dosage form is obtained. Hence, the selected dosage and the selected dosage form shall depend on the desired therapeutic effect, the route of administration and the duration of the treatment. Dosage ranges for the combination may be from the maximal tolerated dose for the single agent to lower doses.

The keratinocytes within the meaning of the present invention include, without being limited to, any of those keratinocytes mentioned hereinabove and hereinbelow.

Preferred keratinocytes of this invention are those disclosed in WO 03/033686, the disclosure of which is incorporated herein.

The invention refers to keratinocytes with a high proliferation potential, which are not immortalised and which can be replicated at least 150 times by in vitro cell culture methods. This results in a cell multiplication factor of about 1044. The corresponding keratinocytes still retain their advantageous properties for the treatment of wounds, such as diabetic wounds (e.g. diabetic foot or ulcus).

The keratinocytes for use according to this invention are primarily keratinocytes isolated from a donor and culturable in vitro, while the isolation and initial cultivation may be carried out by anyone skilled in the art, according to the process described by Rheinwald and Green in 1975, for example.

In this context, the invention preferably refers to keratinocytes which are isolated from the epidermal part of a foreskin. Keratinocytes of human origin, particularly keratinocytes of the culture KC-BI-1, which were deposited on 27 Jun. 2001 at the DSMZ-Deutsche Sammlung von Mikroorganismen and Zellkulturen [German Collection of Microorganisms and Cell Cultures] GmbH, Braunschweig, Germany under Accession Number DSM ACC2514 for the purposes of patent proceedings according to the Budapest Agreement, are preferred for the purpose of this invention. The invention also refers to keratinocytes which are derived from the culture KC-BI-1 (DSM ACC2514). Thus, the invention also refers to all cells and cultures which are and/or may be generated by subpassaging and/or subcloning the original culture KC-BI-1.

The cultivation of the keratinocytes of this invention is described by way of example in relation to the keratinocytes KC-BI-1 (Example 1). The use of the complex medium specified in Example 1 and the use of feeder cells, preferably the use of lethally irradiated murine 3T3 fibroblasts, is advantageous for the culturing. The amount of foetal calf serum should be between 2 and 10%. The preparation of the feeder cells is known to those skilled in the art and may be carried out for example by the process described in Example 2. Subcultivation of the keratinocytes according to the invention at a maximum confluence of 80% is particularly advantageous. The keratinocytes may be cultured at 35 to 38° C., preferably at 37° C., at a relative humidity of >90%, preferably 95% and a CO₂ saturation of 5 to 9%.

With the process described in Example 1 the population doubling time for the keratinocytes, particularly human keratinocytes from the epidermal part of a foreskin, is between 1 and 2 days (FIG. 1). The cells can be cultured over numerous passages at a substantially constant replication rate (FIG. 2). However, the present invention is not restricted to just the keratinocytes KC-BI-1, but rather it contemplates any keratinocytes according to the following embodiments E1-E8:

For example, keratinocytes for use according to the present invention are such keratinocytes according to the following embodiments E1-E8:

E1: Keratinocytes, characterised in that they are not immortalised and may be doubled at least 150 times by in vitro cell culture methods.
E2: Keratinocytes according to embodiment E1, isolated from the epidermal parts of a foreskin.
E3: Keratinocytes according to embodiment E1, characterised in that they are cells from the culture KC-BI-1 (DSM ACC 2514), or keratinocytes derived therefrom.
E4: Keratinocytes according to embodiments E1-E3, wherein said keratinocytes
cannot be replicated in the absence of foetal calf serum and/or
in the absence of feeder cells and/or
in the absence of Epidermal Growth Factor (EGF).
E5: Keratinocytes according to embodiments E1-E4, wherein the said keratinocytes have little or no telomerase activity, preferably in comparison to immortalised keratinocytes, preferably to the cell line HaCaT.
E6: Keratinocytes according to embodiments E1-E5, characterised in that the said keratinocytes can be replicated at least 200 times by in vitro cell culture methods.
E7: Keratinocytes according to embodiments E1-E6, characterised in that the said keratinocytes can be replicated at least 250 times by in vitro cell culture methods.
E8: Keratinocytes according to embodiments E1-E7, characterised in that the said keratinocytes can be replicated at least 300 times by in vitro cell culture methods.

For further example, keratinocytes for use according to the present invention are such keratinocytes according to embodiment F1:

F1: Keratinocytes, characterised in that they are not immortalised and may be replicated at least 150 times by in vitro cell culture methods and that they are cells from the culture KC-BI-1 (DSM ACC 2514), or keratinocytes derived therefrom, wherein said keratinocytes
a. cannot be replicated in the absence of foetal calf serum and/or
b. in the absence of feeder cells and/or
c. in the absence of Epidermal Growth Factor (EGF),
and wherein the telomerase activity of said keratinocytes is lower than that of the cell line HaCaT by at least factor 2,
and wherein derived keratinocytes are cells and cultures which are and/or may be generated by subpassaging and/or subcloning the original culture KC-BI-1 (DSM ACC 2514).

In certain embodiments, the combinations, compositions, methods and uses according to this invention relate to combinations wherein the DPP-4 inhibitor and the keratinocytes are preferably selected according to the entries in the Table 1:

TABLE 1

| DPP-4 inhibitor | keratinocytes |
|---|---|
| linagliptin | selected from embodiment E1 |
| linagliptin | selected from embodiment E2 |
| linagliptin | selected from embodiment E3 |
| linagliptin | selected from embodiment E4 |
| linagliptin | selected from embodiment E5 |
| linagliptin | selected from embodiment E6 |
| linagliptin | selected from embodiment E7 |
| linagliptin | selected from embodiment E8 |
| linagliptin | selected from embodiment F1 |
| linagliptin | cells from KC-BI-1, or |
| | derived therefrom |

The expression “not immortalised” in relation to the present invention means that primarily isolated keratinocytes and the keratinocytes cultured here are not spontaneously transformed and/or have not been transformed by molecular-biological, chemical or physical methods known from the research. The latter means that the cells have not been treated either using e.g. viral factors or sequences, chemically mutagenic substances or, for example, by irradiation or a combination of different processes.

Keratinocytes which “are not immortalised” also means that compared with transformed tumour cell lines (Harle-Bachor and Boukamp, 1993) or compared with immortalised cell lines, preferably the cell line HeLa, the said keratinocytes have no telomerase activity or a substantially reduced telomerase activity (cf. FIG. 3). This is particularly true also in comparison with the immortalised keratinocyte cell line HaCat.

The expression “not immortalised” also means that said keratinocytes cannot be replicated in the absence of foetal calf serum and/or in the absence of feeder cells and/or in the absence of epidermal growth factor, EGF, as immortalised keratinocytes can, for example (Schoop et al., 1999).

The expression “not immortalised” also means, however, that said keratinocytes do not change their characteristic phenotype as the cell replication increases (cf. FIG. 4).

“Not immortalised” also means that the said keratinocytes exhibit a normal differentiation profile after transplantation onto nude mice, preferably BALB/c. “Normal differentiation potential” here means the ability of the keratinocytes to develop into terminally differentiated keratinocytes and form suprabasal epidermal layers as well as a stratum corneum in the same way as autologous keratinocytes.

The present invention also refers to keratinocytes which are not immortalised and which can be replicated at least 200 times by in vitro cell culture methods. The invention further refers to keratinocytes which are not immortalised and can be replicated at least 250 times by in vitro cell culture methods. The invention also refers to keratinocytes which are not immortalised and can be replicated at least 300 times by in vitro cell culture methods.

These keratinocytes can be replicated from only one donor or only a few donors. Starting from one donation, for example, 10⁴⁴ cells may be produced after 150 cell replications, 10⁷⁷ cells after 250 cell replications and 10⁹⁰ cells after 300 cell replications. Thus, it is possible to produce large quantities of standardised cell material for the preparation of biologically active wound healing aggregates of constant, verifiable quality. A corresponding amount of standardised cell material may be replicated, for example, starting from a cryopreserved cell bank, which is in turn produced from keratinocytes having the properties according to the invention.

By using standardised cell material as the starting material for preparing biologically active wound healing dressings the risk of infection to the possible recipients is also reduced, as the isolation of the keratinocytes is restricted to a few donors, preferably one donor.

Thus, it is overcome serious disadvantages which currently consist of the merely restricted passaging of prior keratinocytes.

The present invention further relates to a pharmaceutical composition or product which comprises a carrier coated with the keratinocytes referred to herein or mentioned herein as keratinocytes of the invention. “Coated” for the purposes of the invention means that the surface of the carrier is partially or totally colonised with the keratinocytes of this invention. A partially colonised carrier is particularly suitable, as a shorter culture time is needed before the carrier can be used for wound treatment.

A suitable carrier for the keratinocytes of the invention is characterised in that it is a biocompatible carrier material which may be pharmaceutically acceptable (e.g. which may be used to prepare a pharmaceutical composition). Hydrophobic biocompatible carrier materials, as described in WO 91/13638, for example, may be used. However, it is also possible to use carrier materials with predominantly hydrophilic properties.

A preferred embodiment of the present invention comprises the use of such carrier materials which contains a polymer of esterified hyaluronic acid (such as e.g. benzyl esterified hyaluronic acid). In a particularly preferred embodiment a polymer of esterified hyaluronic acid is used, consisting of a perforated polymer film of a defined geometry. The polymer film has a thickness of 10 to 500 μm, for example, and is perforated with holes measuring between 10 and 1000 μm, the holes being of a defined, constant size and forming ordered rows, separated from one another by a constant spacing of 50 to 1000 μm. A film of this kind is described in EP 0 462 426. Perforated carrier materials are particularly suitable as they do not require the biologically active wound dressing to be placed on the wound in any particular direction. Example 3 describes the preparation, by way of example, of a perforated carrier matrix of esterified hyaluronic acid of a defined geometry colonised by keratinocytes of the invention. The carrier matrix is a product made by Messrs Fidia Advanced Biopolymers Ltd., Abano Terme, Italy, marketed in Germany under the product name “Laserskin”. The particular suitability of this carrier material for producing a biologically active wound dressing in conjunction with keratinocytes has already been demonstrated on an animal model (Lam et al., 1999) and in humans (Harris et al., 1999). Apart from improved migration and differentiation of the epithelial cells the matrix consisting of hyaluronic acid ester has a positive effect on angiogenesis and collagen production. The wound dressings also used with hyaluronic acid ester as matrix are, however, coated with autologous keratinocytes and/or skin equivalents of complex structure obtained from keratinocytes and fibroblasts. They therefore suffer from some disadvantages. These disadvantages may be overcome particularly by the use of the advantageous allogeneic keratinocytes as described herein.

Another embodiment of the invention relates to a pharmaceutical composition or product comprising the keratinocytes of this invention together with reabsorbable polymers as carriers, e.g. one or more selected from polyesters, polycarbonates, polyanhydrides, polyorthoesters, polydepsipeptides, polyetheresters, polyamino acids or polyphosphazenes, especially poly(L-lactide), poly(D,L-lactide), poly(L-lactide-co-D,L-lactide), poly(glycolide), poly(L-lactide-co-glycolide), poly(L-lactide-co-trimethylene-carbonate) and/or poly(dioxanone). These polymers can be both perforated and unperforated.

For example, pharmaceutical compositions or products containing keratinocytes of the present invention are such compositions or products according to the following embodiments E9-E13:

E9: Pharmaceutical composition or product comprising, consisting or consisting essentially of a carrier which is coated with keratinocytes according to one of embodiments E1-E8,
a. wherein the carrier is partially colonised with keratinocytes; or
b. wherein the carrier is completely colonised with keratinocytes.
E10: Pharmaceutical composition or product according to embodiment E9, characterised in that the carrier is a biocompatible carrier material, which may be pharmaceutically acceptable.
E11: Pharmaceutical composition or product according to embodiment E10, characterised in that the carrier material is a hydrophobic or hydrophilic biodegradable membrane.
E12: Pharmaceutical composition or product according to embodiment E10 or E11, characterised in that the carrier is a polymer of esterified hyaluronic acid, preferably a perforated polymer film of defined geometry,
a. wherein the polymer film has a thickness of 10 to 500 μm and is perforated with holes measuring between 10 and 1000 μm, the holes having a defined, constant size and forming an ordered row, in which they are separated from one another by a constant spacing of 50 to 1000 μm.
E13: Pharmaceutical composition or product according to embodiment E10 or E11, characterised in that the carrier material is polyester, polycarbonates, polyanhydrides, polyorthoesters, polydepsipeptides, polyetheresters, polyamino acids or polyphosphazenes,
a. particularly poly(L-lactide), poly(D,L-lactide), poly(L-lactide-co-D,L-lactide), poly(glycolide), poly(L-lactide-co-glycolide), poly(L-lactide-co-trimethylene-carbonate) or poly(dioxanone),
b. wherein the said polymers are perforated or
c. not perforated.

For further example, compositions or products containing keratinocytes of the present invention are such compositions or products according to the following embodiments F2-F6:

F2: Pharmaceutical composition or product comprising, consisting or consisting essentially of a carrier which is coated with keratinocytes according to embodiment F1,
a. wherein the carrier is partially colonised with keratinocytes; or
b. wherein the carrier is completely colonised with keratinocytes.
   F3: Pharmaceutical composition or product according to embodiment F2, characterised in that the carrier is a biocompatible carrier material, which may be pharmaceutically acceptable.
   F4: Pharmaceutical composition or product according to embodiment F2, characterised in that the carrier material is a hydrophobic or hydrophilic biodegradable membrane.
   F5: Pharmaceutical composition or product according to embodiment F3 or F4, characterised in that the carrier is a polymer of esterified hyaluronic acid, preferably a perforated polymer film of defined geometry, wherein the polymer film has a thickness of 10 to 500 μm and is perforated with holes measuring between 10 and 1000 μm, the holes having a defined, constant size and forming an ordered row, in which they are separated from one another by a constant spacing of 50 to 1000 μm.
   F6: Pharmaceutical composition or product according to embodiment F3 or F4, characterised in that the carrier material is polyester, polycarbonates, polyanhydrides, polyorthoesters, polydepsipeptides, polyetheresters, polyamino acids or polyphosphazenes,
a. particularly poly(L-lactide), poly(D,L-lactide), poly(L-lactide-co-D,L-lactide), poly(glycolide), poly(L-lactide-co-glycolide), poly(L-lactide-co-trimethylene-carbonate) or poly(dioxanone),
b. wherein the said polymers are perforated or
c. not perforated.

Further, the present invention relates to a pharmaceutical composition or product (e.g. a pharmaceutical composition for topical use or a biologically active wound dressing, BAWD) containing the keratinocytes of the present invention (e.g. such keratinocytes according to at least one embodiment E1 to E8 or F1) together with a suitable carrier (e.g. a perforated esterified hyaluronic acid polymer matrix), and a DPP-4 inhibitor, particularly linagliptin.

Further, the present invention relates to a pharmaceutical composition or product (e.g. a pharmaceutical composition for topical use or a biologically active wound dressing, BAWD) containing a pharmaceutical composition or product consisting of a carrier (e.g. as described herein) which is coated with the keratinocytes of the present invention (e.g. such keratinocytes according to at least one embodiment E1 to E8 or F1), and a DPP-4 inhibitor, particularly linagliptin.

Further, the present invention relates to a pharmaceutical composition or product (e.g. a pharmaceutical composition for topical use or a biologically active wound dressing, BAWD) containing a pharmaceutical composition or product according to at least one embodiment E9 to E13 or F2 to F6, and a DPP-4 inhibitor, particularly linagliptin.

Further, the present invention relates to a pharmaceutical combination, composition or product (e.g. a pharmaceutical composition for topical use or a biologically active wound dressing, BAWD) comprising, consisting, or consisting essentially of
a) keratinocytes of the present invention (e.g. such keratinocytes according to at least one embodiment E1 to E8 or F1) and optionally a pharmaceutically acceptable carrier (e.g. esterified hyaluronic acid matrix), such as e.g. a pharmaceutical composition or product according to at least one embodiment E9 to E13 or F2 to F6, and
b) a DPP-4 inhibitor, particularly linagliptin.

Further, the present invention relates to a kit or product including

a) a pharmaceutical composition or product containing keratinocytes of the present invention (e.g. such keratinocytes according to at least one embodiment E1 to E8 or F1), and optionally one or more pharmaceutically acceptable carriers, and
b) a pharmaceutical composition or product containing a DPP-4 inhibitor, particularly linagliptin, and optionally one or more pharmaceutically acceptable carriers.

There is also provided a method of cryopreserving the keratinocytes of the invention at a temperature of −20° C. to −196° C., preferably at a temperature below −180° C. The said keratinocytes can be frozen by standardised methods familiar to anyone skilled in the art. DMSO, inter alia, may be used as the cryoprotectant. It is also possible to use other cryoprotectants such as glycerol, hydroxyethyl starch or a combination of the two, and a combination of these with DMSO. Suitable methods are described for example in WO 96/24018, U.S. Pat. No. 5,891,617 or U.S. Pat. No. 5,298,417.

There is also provided cryopreserving of the carriers coated with the keratinocytes of the invention, characterised in that the keratinocytes with their corresponding carrier are cryopreserved at a temperature of −20° C. to −196° C., preferably at −60° C. to −80° C. The advantage of cryopreserving is that the product obtained in large quantities can be stored and thus examined for uniformity of quality by random sampling before clinical use. Finally, storage ensures that the wound healing aggregates are available at short notice for medical purposes.

A suitable cryoprotectant for such keratinocytes-containing product of the invention is hydroxyethyl starch, for example, in a concentration of 7-13% (w/w). However, it is also possible to use DMSO or glycerol as well as a combination of various cryoprotectants, particularly hydroxyethyl starch, DMSO and/or glycerol. It is also possible to use trehalose as cryoprotectant.

After a rapid reduction in temperature from 37° C. to −5 to −10° C., preferably −6 to −8° C. within 2-5 min, the product comprising carrier and keratinocytes of the invention is equilibrated at the appropriate temperature for 15-30 min, preferably for 23-26 min. Then the product is cooled at a freezing rate of <1° C./min, preferably from 0.2 to 0.6° C./min, most preferably 0.4° C./min, to a temperature of e.g. −60 to −80° C.

Example 4 describes, by way of example, the cryopreserving of a carrier matrix of hyaluronic acid ester coated with KC-BI-1. However, it is also possible to use other methods of cryopreserving, e.g. the methods described in WO 95/707611, WO 96/24018, EP 0 296 475; this list should not be regarded as exhaustive but merely indicates that methods of cryopreserving products consisting of biocompatible carriers and keratinocytes are part of the current state of the art.

The present invention further relates to the pharmaceutical compositions or products according to this invention for use in treating wounds, such as e.g. diabetic wounds (e.g. diabetic foot or ulcer), and/or burns.

The present invention further relates to the use of the pharmaceutical compositions or products according to this invention for preparing medicaments for use in treating wounds, such as e.g. diabetic wounds (e.g. diabetic foot or ulcer), and/or burns.

The present invention also relates to the medical use of the keratinocytes of the invention described here, optionally in combination with a DPP-4 inhibitor, particularly linagliptin, and/or the composition or product of said keratinocytes with a carrier option described here, optionally in combination with a DPP-4 inhibitor, particularly linagliptin, particularly to their use for treating wounds, such as e.g. diabetic wounds (e.g. diabetic foot or ulcer).

One embodiment of the invention refers to the use of the keratinocytes of the invention and/or the composition or product of said keratinocytes with a carrier in the treatment of burns and/or ulcers, optionally in combination with a DPP-4 inhibitor, particularly linagliptin.

Burns which may be treated are preferably second degree burns while the ulcers are preferably chronic ulcers of the lower leg which are difficult to heal, of the type Ulcus cruris, preferably Ulcus cruris venosum or diabetic ulcers, and also decubital ulcers.

The medical use includes a combined and/or supplementary use of the active compounds, combinations, compositions or products according to the invention with conventional therapies known in the art with a beneficial effect on wound healing. This means a combined and/or supplementary use of one or more other substance(s) with a beneficial effect on wound healing. Mention may be made of the supplementary and/or combined treatment of Ulcus cruris venosum with hydrocolloid dressings and/or the additional use of antimicrobial substances, e.g. the administering of antibiotics. Further mention may be made of the treatment of diabetic wounds using Regranex™ (human recombinant platelet derived growth factor in carboxymethyl cellulose), in combination; or using NorLeu³-angiotensin (1-7), in combination.

The invention also relates to the combinations, compositions or products according to the invention containing at least the keratinocytes of the invention and/or the composition or product of a biocompatible carrier and said keratinocytes for preparing a medicament for treating wounds, such as for treating burns and/or ulcers, e.g. for the treatment of second degree burns, Ulcus cruris (venosum), diabetic ulcers or decubital ulcers; particularly diabetic wounds (e.g. diabetic foot or ulcer), optionally in combination with a DPP-4 inhibitor, particularly linagliptin.

The invention further relates to a process for treating these wounds, this process being characterised in that the keratinocytes of the invention and/or the product of the invention comprising keratinocytes and carrier is or are placed on the wounds to be treated, optionally in combination with a DPP-4 inhibitor, particularly linagliptin. The keratinocytes and the product may be used either fresh or after cryopreservation. A corresponding method of treating wounds is described in Example 5.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1: Cell replication of the keratinocytes KC-BI-1 as a function of the culture time. This shows the number of cell replications (CPD=Cumulative Population Doublings) of the keratinocytes KC-BI-1 over a culture period of 94 days. Within the 94 day observation period the cells doubled roughly 75 times. This corresponds to a mean doubling time of 1.25 days per cell replication, or 12.5 per 10 days.

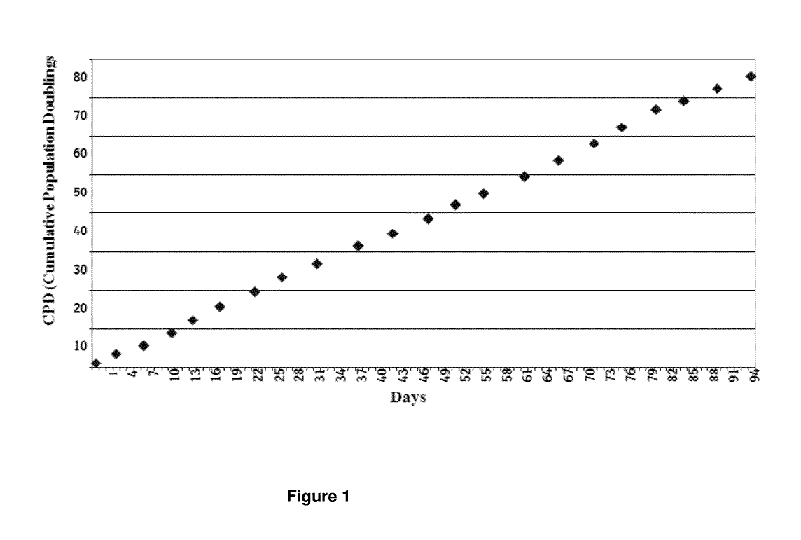

FIG. 2: Doubling of the keratinocytes KC-BI-1 over a period of 10 months. This shows the cell replication of the keratinocytes KC-BI-1 over 10 months, given as population doublings (PD) as a function of the cell passages 1-67.

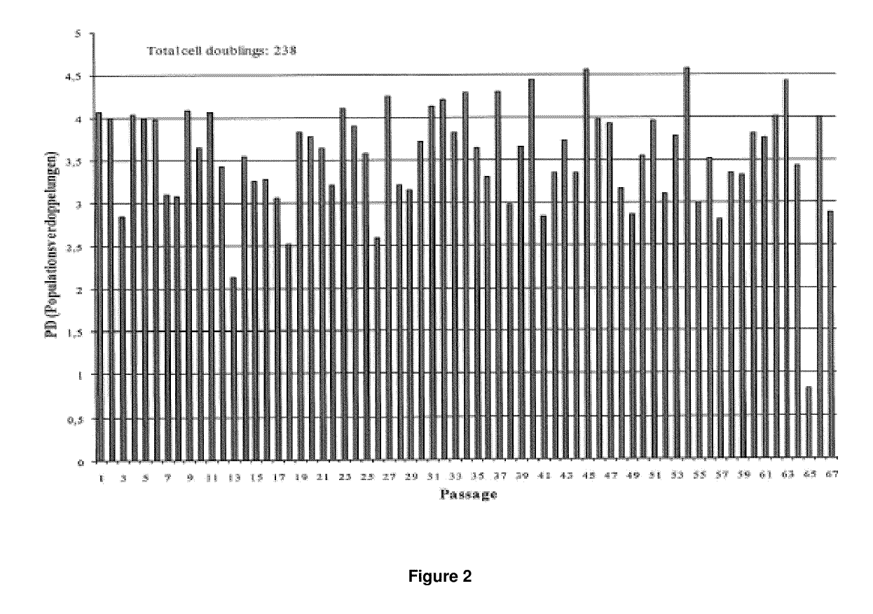

FIG. 3: Determining the relative telomerase activity. This shows the relative telomerase activity for the keratinocytes KC-BI-1 after passage 1, 12, 18, 40 and 57 compared with the activity of the cell line HeLa. The Figure shows almost no or only slight telomerase activity for the keratinocytes KC-BI-1 compared with the immortalised cell line HeLa.

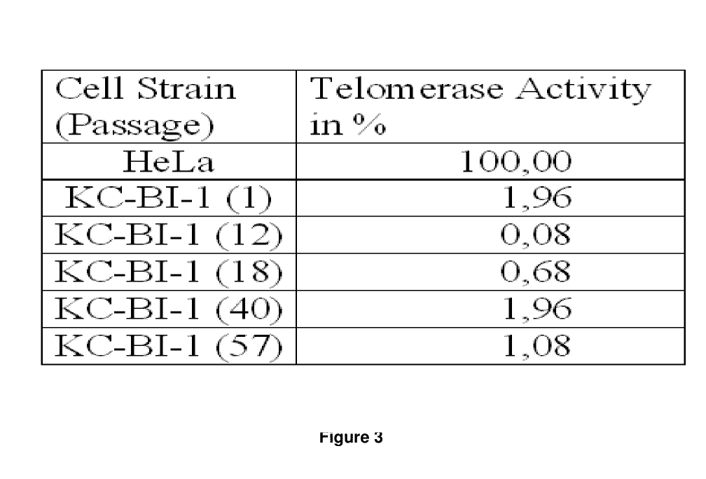

FIG. 4: Morphology of the keratinocytes KC-BI-1 after passages 5 and 60. Viewed under the optical microscope the keratinocytes KC-BI-1 do not exhibit any morphological differences between cell passage 5 (culture time 25 days) and cell passage 60 (culture time 300 days).

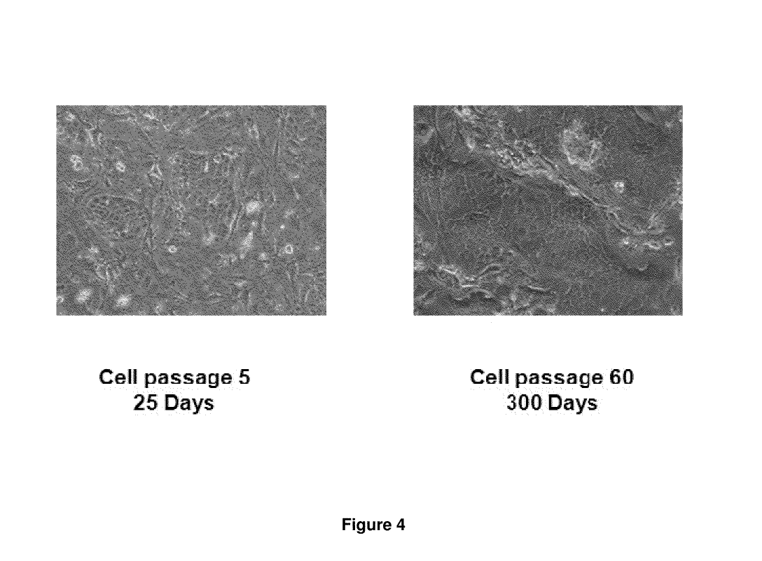

FIG. 5 shows the induction of robust granulation tissue in wounds of diabetic mice by application of a keratinocyte-containing hyaluronic acid matrix (compared to hyaluronic acid matrix alone). Blue: collagen staining; red: nucleic and cytoplasmic staining; red arrows: epithilial margins.

### EXEMPLIFYING EMBODIMENTS

### Example 1

### Process for Culturing the Keratinocytes According to the Invention Taking the Culture KC-BI-1 (DSM ACC2514) as an Example

### 1. Material

Keratinocytes KC-BI-1; irradiated 3T3-murine fibroblasts (feeder cells, for preparation cf. Example 2); cell culture medium K/1 (for composition see below); EDTA (0.02%); trypsin/EDTA (0.05%/0.01%); cell culture flasks (T-flask): 25 cm², 80 cm², 175 cm²

### 2. Thawing the Cells

### 2.1 Thawing the Feeder Cells

The cells are rapidly thawed and placed in 5-10 ml of preheated K/1 medium. A corresponding quantity of feeder cells are transferred into a suitable cell culture flask and topped up with K/1 medium:

| | |
|---|---|
| 25 cm² T-flask 0.5 × 10⁶ cells | Final volume of medium: 5-6 ml |
| 80 cm² T-flask 1.5 × 10⁶ cells | Final volume of medium: 20 ml |
| 175 cm² T-flask 3.5 × 10⁶ cells | Final volume of medium: 50 ml |

The feeder cells may be used immediately or within 24 hours.

### 2.2 Thawing the Keratinocytes

The cells are rapidly thawed and placed in 5-10 ml of preheated K/1 medium. A corresponding quantity of cells are added to the cell culture flasks already containing feeder cells and topped up with fresh medium:

25 cm² T-flask 0.15×10⁶ cells; Final volume of medium: 6-10 mL
80 cm² T-flask 0.4×10⁶ cells; Final volume of medium: 20 mL
175 cm² T-flask 1×10⁶ cells; Final volume of medium: 50 mL

### 3.0 Cultivation

The cells are incubated at 35-39° C., preferably at 37° C. The relative humidity is >90%, preferably 95% and the CO₂ concentration is 5-9%. The keratinocytes are subcultured at a maximum confluence of 80%.

For this, the cell culture supernatant is discarded. The feeder cells are rinsed twice with 0.02% EDTA (2-10 ml) and incubated for 5-10 min at 37° C., then detached from the cell culture flask by tapping it (or shaking it). The keratinocytes are then treated with trypsin/EDTA (0.05%/0.01%, 1-6 ml) for 5-10 min at 37° C. and carefully detached by tapping. If necessary the remaining cells are carefully scraped off using a cell spatula. The trypsin/EDTA solution is neutralised by the addition of medium and the cells are separated by careful pipetting up and down. The cells are seeded out in the cell numbers specified in 2.2. The cell culture medium is changed on day 3 and then every two days.

### 4.0 Preparation of the K/1 Medium

All the components and the stock solutions are combined one after another in the sequence given in Table 1. The mixture is made up to 1 litre with WFI (water for injection). Then the pH is adjusted to 7.0 to 7.2 with NaOH or HCl. The osmolarity should be between 320-400 mOsm/kg. Finally, the medium is sterile-filtered.

### 4.1 Preparation of the Stock Solutions

### Triiodothyronine

13.6 mg of triiodothyronine are dissolved in 1 ml of 0.1 NaOH and 99 ml of PBS are added thereto. The finished solution is diluted 1:100 in PBS. 1 ml of this solution is required per litre of medium.

### EGF

1 mg of EGF is dissolved in 100 ml of WFI. 1 ml of this solution is required per litre of medium.

rh-Insulin (Only Soluble at pH<4.0)

5 g of rh-insulin are added to 0.9 L of WFI and the pH is adjusted to 2.5 with 6M HCl. After the rh-insulin has dissolved, the pH is adjusted to 8.0 with 1M NaOH. The mixture is made up to 1 litre with WFI. 1 ml of this solution is required per litre of medium.

### 4.2 Composition of the Medium

| components | concentration/L | |
|---|---|---|
| WFI (water for injection) | 0.8 | L |
| DMEM with glutamine | 10.035 | g |
| HAM's F12 | 2.66 | g |
| sodium hydrogen carbonate | 3.07 | g |
| Na-pyruvate | 0.11 | g |
| Apo-Transferrin | 5 | mg |
| Adenine-Hemisulphate | 147.4 | mg |
| Forskolin (soluble in a few drops of DMSO) | 2.05 | mg |
| Rh-Insulin-Concentrate | 1.0 | mL |
| Hydrocortisone 10 mM | 0.11 | mL |
| triiodothyronine stock solution | 1.0 | mL |
| EGF-stock solution | 1.0 | mL |
| Phenol red | 8.1 | mg |
| FCS (2-10%) | 20-100 | mL |
| 6M HCl | as required | |
| 40% NaOH | as required | |
| WFI | add to 1.0 | L |

However, the cells may also be cultured from fresh biopsy material, e.g. from the epidermal part of a foreskin. The primary isolation of the undifferentiated, proliferating keratinocytes may be carried out using the method described by Rheinwald and Green in 1975.

The feeder cells used may be, for example, the cells described in Example 2. It is also conceivable to use other lethal fibroblasts, preferably other murine fibroblasts, most preferably descendants of cell line 3T3.

### Example 2

### Preparation of Irradiated 3T3 Feeder Cells for Cultivating Keratinocytes

### 1. Material

Murine 3T3 fibroblasts (e.g. ATCC CCL 92, 3T3-Swiss albino, contact-inhibited fibroblasts) which may be obtained from the American Type Culture Collection (ATCC), 10801 University Boulevard, Manassas, Va., USA, DMEM+10% foetal calf serum (FCS); PBS; 0.2% trypsin solution; 0.04% EDTA solution; cell culture flasks (T-flasks): 25 cm², 80 cm², 175 cm²

### 2. Thawing the Cells

The cells are rapidly thawed and added to 5-10 ml of preheated medium. DMSO-containing medium is removed after centrifugation. The cells are suspended in 5-10 ml of medium. After the cell number has been determined the cells are seeded into suitable cell culture flasks in a density of 10³ to 104 cells/cm². They are incubated at 35-39° C., preferably at 37° C. The relative humidity is >90%, preferably 95% and the CO₂ concentration is 5-9%.

### 3. Culturing the Cells

The cells are inspected daily for growth. The cell density should not exceed a maximum confluence of 70-80%. Subculturing is carried out, as necessary, every 2 to 4 days. For this, the medium is discarded and the cells are washed with a suitable amount of a 1:2 mixture of EDTA and trypsin (0.5-5 ml). Then the cells detached are taken up in 3.5-20 ml of medium. The cells are re-seeded at a density of 10³ to 10⁴ cells/cm².

### 4. Irradiation of the Feeder Cells

The feeder cells are irradiated with a dose of about 60 Gy (6000 rad in a ¹³⁷Cs source).

Both the irradiated and the non-irradiated cells can be cryopreserved in liquid nitrogen using standard methods and stored for long periods.

### Example 3

### Method of Coating a Carrier Matrix, in this Case Laserskin, with Keratinocytes from the Culture KC-BI-1 (DSM ACC2514)

The preparation of the biologically active wound healing dressing according to the invention will now be described by way of example. The wound healing dressing described here consists of the keratinocytes KC-BI-1 according to the invention and Laserskin, a bioreabsorbable carrier matrix of hyaluronic acid ester.

However, the invention is not restricted to the combination described here. Rather, any keratinocytes which have the novel properties recited in claims 1 to 8 may be used for the coating.

It is also possible to use other suitable carrier matrices, provided that they are biocompatible carrier materials which may be used to prepare a pharmaceutical composition. For example, hydrophobic biocompatible carrier materials as described in WO 91/13638 may be used. In addition, however, it is also possible to use carrier materials with predominantly hydrophilic properties.

Another preferred embodiment of the invention comprises using the keratinocytes according to the invention together with reabsorbable polymers, consisting of polyesters, polycarbonates, polyanhydrides, polyorthoesters, polydepsipeptides, polyetheresters, polyamino acids or polyphosphazenes, especially poly(L-lactide), poly(D,L-lactide), poly(L-lactide-co-D,L-lactide), poly(glycolide), poly(L-lactide-co-glycolide), poly(L-lactide-co-trimethylene-carbonate) or poly(dioxanone), and using perforated films consisting of said polymers.

### 1. Material

K/1 medium (cf. Example 1); PBS, 0.04% EDTA (diluted to 0.02% with PBS); trypsin/EDTA (0.05%/0.01%); sterile Roux dishes (T 25 cm², T 80 cm², T 175 cm²), Laserskin (Messrs. Fidia Advanced Biopolymers srl, Abano Terme, Italy) in 144×21 Petri dishes (145 cm² surface area); 3T3 feeder cells; keratinocytes according to the invention such as KC-BI-1 for example

### 2. Culturing the Biologically Active Wound Healing Dressing

### 2.1. Material

Irradiated feeder cells, e.g. the murine 3T3 fibroblasts mentioned in Example 2; keratinocytes according to the invention from stock; 8.5 cm×8.5 cm pieces of Laserskin in a Petri dish (finished product); K/2 medium

### 2.2. Seeding Out the 3T3 Feeder Cells

The feeder cells, prepared according to Example 2, are placed on Laserskin in a seeding density of about 15,000 to 25,000 cells/cm² (roughly corresponding to 3×10⁶ cells/Petri dish). The Petri dish is then incubated at 35 to 37° C. at >90% relative humidity and 5-11% CO₂, preferably 7-9%, in an incubator at 37° C. The keratinocytes are seeded onto the feeder cell lawn either on the same day or, at the latest, the next day (after 24 hours).

### 2.3. Seeding Out and Culturing the Keratinocytes

The biologically active wound healing dressings are prepared with the keratinocytes according to the invention. The subculturing of the keratinocytes may, for example, be carried out as follows:

The subconfluent cultures are rinsed once with 0.02% EDTA (80 cm² Roux dish: 8 mL; 175 cm² Roux dish: 10 ml). Then the feeder cells are incubated with 0.02% EDTA for 5-10 min at 37° C. (80 cm² Roux dish: 8 mL; 175 cm² Roux dish: 10 ml) and detached by shaking carefully.

The keratinocytes are dissolved as in Example 1 with a trypsin/EDTA mixture (0.05%/0.01%) (80 cm² Roux dish: 2-3 mL; 175 cm² Roux dish: 5-6 ml), then taken up in cell culture medium (80 cm² Roux dish: 7-8 mL; 175 cm² Roux dish: 14-15 ml) and separated by carefully pipetting up and down.

The keratinocytes according to the invention are applied to the Laserskin film provided with feeder cells, in a seeding density of about 15,000 to 25,000 cells/cm² (roughly corresponding to 3×10⁶ cells/Petri dish). Then the cells are incubated until 30-100% confluent, preferably 80-100% confluent, at 35-39° C., preferably at 37° C. The relative humidity is >90%, preferably 95% and the CO₂ concentration is 5-9%.

### Example 4

### Method of Cryopreserving the Biologically Active Wound Healing Dressing According to the Invention

After the keratinocytes have colonised the carrier matrix to a confluence of 30-100%, preferably 80-100%, the product according to the invention may be frozen in suitable containers, e.g. heat-sealable PP bags, under controlled conditions. To do this, culture medium is carefully removed and replaced by 20 ml of K/2 freezing medium at a temperature of 2-6° C. The product is then packaged under sterile conditions and frozen according to the following procedure:

After a rapid lowering of the temperature to −5 to −10° C., preferably −6 to −8° C. within 2-5 min, the product is equilibrated at the corresponding temperature for 15-30 min, preferably for 23-25 min Then the product is cooled to a temperature of, for example, −60 to −80° C. at a freezing rate of <1° C./min, preferably 0.2 to 0.6° C./min, most preferably 0.4° C./min The product is stored at −60 to −80° C.

### K/2 Freezing Medium:

K/1 growth medium (cf. Example 1) mixed with 7-13% (w/w) of hydroxyethyl starch.

### Example 5

### Example of the Use of a Carrier Matrix Colonised with the Keratinocytes According to the Invention for Covering Wounds, Taking Venous Leg Ulcers as an Example

### 1. Transporting Freshly Prepared Wound Healing Dressings

After the keratinocytes have grown to 30-100%, preferably 80-100% confluence on the Laserskin, the culture is rinsed one or more times with a suitable quantity, preferably 30 ml, of K/3 transporting medium. The biologically active wound healing dressing is transported in a suitable amount, preferably in 20 ml, of K/3 transporting medium. The headroom of the Petri dish is briefly gassed with an air mixture of 5-10% CO₂, sealed with adhesive tape, e.g. Parafilm, and immediately delivered to the clinic in a transportation box.

### K/3 Transporting Medium:

Growth medium K/1 (cf. Example 1) without foetal calf serum (FCS). However, it is also possible to use simple physiological saline solutions e.g. based on phosphate-borate, e.g. PBS, or based on HEPES (N-2-hydroxyethylpiperazin-N′-2-ethanesulphonic acid) or MES ([2-N-morpholino]ethanesulphonic acid).

### 2. Transporting Cryopreserved Wound Healing Dressings

Cryopreserved wound healing dressings may typically be supplied to the clinics on dry ice. However, other forms of transportation are possible, provided that the wound healing dressings are transported at a temperature below −60° C.

The cryopreserved wound healing dressings are rapidly thawed. Then the freezing medium is removed and the dressing is rinsed one or more times with K/3 transporting medium (see above) or another suitable physiological solution such as Ringer's solution, for example.

### 3. Therapeutic Use

The dressing is then placed on the wound. When non-perforated carrier materials are used, the wound healing dressing has to be positioned correctly with the cells facing the wound. The use of perforated carriers which allow keratinocytes to colonise both sides of the carrier (e.g. Laserskin) means that the wound healing dressing according to the invention does not have to be placed on the wound being treated in any particular direction. Depending on the success of the therapy the treatment may be repeated a number of times.

### Example 6

### Genetic Characterisation of the Keratinocyte Cell KC-BI-1 (DSM ACC2514)

KC-BI-1 cells were subcultured over a number of passages using the method according to the invention described above. Cells from passage 4, 13 and 121 were then subjected to genetic analysis, investigating the length polymorphism of 15 different loci (CSF1PO, D13S317, D16S539, D18S51, D21S11, D3S1358, D5S818, D7S820, D8S1179, FGA, Penta D, Penta E, TH01, TPDX and vWA). Analysis was carried out using a method known in the art. For this, the corresponding alleles were amplified using a test to determine paternity (PoewerPlex® 16 System) produced by Messrs Promega (Mannheim, Germany), according to the manufacturer's instructions. The alleles may be identified by determining the fragment length (length standard ILS 600 is part of the above kit). Data on allele frequencies in the population can be found in the corresponding Tables.

Analysis has shown agreement of all the alleles at all the loci for all the cell passages analysed. The data thus enable the KC-BI-1 cells to be genetically classified. A classification probability of >99.999% was determined from the allele frequencies.

### Determining the DNA Length Polymorphism:

| | | KC-BI-1 | KC-BI-1 | KC-BI-1 |
|---|---|---|---|---|
| Marker | Donor | Passage 4 | Passage 13 | Passage 121 |
| CSF1PO | 11 | 11 | 11 | 11 |
| | 12 | 12 | 12 | 12 |
| D13S317 | 8 | 8 | 8 | 8 |
| | 13 | 13 | 13 | 13 |
| D16S539 | 11 | 11 | 11 | 11 |
| | 13 | 13 | 13 | 13 |
| D18S51 | 13 | 13 | 13 | 13 |
| | 17 | 17 | 17 | 17 |
| D21S11 | 29 | 29 | 29 | 29 |
| | 29 | 29 | 29 | 29 |
| D3S1358 | 15 | 15 | 15 | 15 |
| | 15 | 15 | 15 | 15 |
| D5S818 | 9 | 9 | 9 | 9 |
| | 11 | 11 | 11 | 11 |
| D7S820 | 9 | 9 | 9 | 9 |
| | 9 | 9 | 9 | 9 |
| D8S1179 | 10 | 10 | 10 | 10 |
| | 14 | 14 | 14 | 14 |
| FGA | 23 | 23 | 23 | 23 |
| | 26 | 26 | 26 | 26 |
| Penta D | 11 | 11 | 11 | 11 |
| | 12 | 12 | 12 | 12 |
| Penta E | 10 | 10 | 10 | 10 |
| | 12 | 12 | 12 | 12 |
| TH01 | 8 | 8 | 8 | 8 |
| | 9.3 | 9.3 | 9.3 | 9.3 |
| TPOX | 8 | 8 | 8 | 8 |
| | 10 | 10 | 10 | 10 |
| vWA | 16 | 16 | 16 | 16 |
| | 18 | 18 | 18 | 18 |

Biologically Active Wound Dressing Improves Wound Healing in Diabetic db/db Mice

Diabetic ulcers are a significant clinical complication of diabetes and treatment options are limited. Biologically Active Wound Dressing (BAWD) consists of the pharmacologically active human keratinocytes (KCBI 1) and the carrier Laserskin™ (Fidia), a perforated hyaluronic acid matrix. KCBI 1 is an extensively-characterized, safe, and highly proliferative keratinocyte cell strain, isolated from human foreskin:
Single cell suspensions of KCBI 1 were cultured onto a matrix of 60 Gy lethally-irradiated 3T3 feeder layers in a standard keratinocyte medium containing supplements such as EGF, adenine, insulin, hydrocortisone, forskolin, and fetal calf serum. Cell seeds for keratinocytes and feeder cells were 6000/cm² and 10.000/cm², respectively. Cells were cultured for 7 days on Laserskin matrix until confluency was achieved
Prior to the experiment, 8-mm punches were made and applied with the cell layer on top into the wounds. They were covered with Cuticerin (Beiersdorf) and Tegaderm (3 M Medica) Animal Wounding
Female C57BL/6J-db/db mice (from Charles River Wiga, Sulzfeld, Germany), aged 12 weeks, were caged individually, monitored for body weight, and wounded: mice were anesthetized, and 6 full-thickness wounds (5 mm in diameter, 3-4 mm apart) were made on the back of each mouse³
Skin biopsy specimens were obtained from the animals on Days 3 and 10 after injury
All animal experiments were performed according to the guidelines and approval of the local Ethics Animal Review Board

### Treatment of Mice

Each experimental group consisted of 7 individual db/db mice
Immediately after wounding, each mouse received coverage of back skin wounds using hyaluronic acid matrix alone (2 posterior wounds), hyaluronic acid plus human keratinocyte layer (BAWD, 2 medial), or no coverage (2 anterior wounds). Wound tissues were isolated from sacrificed mice on Days 3 and 10 after surgery
Blood glucose levels of all db/db mice were >400 mg/dL and did not change over time

Wounds were isolated and analysed histologically (immunohistochemistry) and by direct RNA sequencing at Day 10 postinjury.

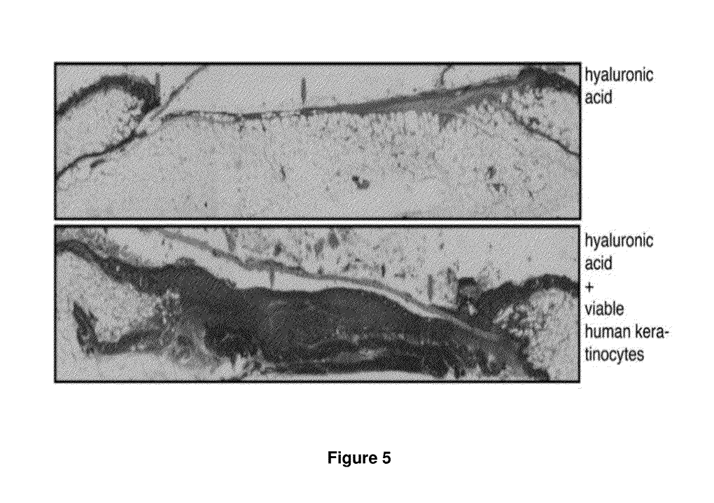

Wounds treated with BAWD exhibited a prominent advancement in wound closure. The wounds showed re-epithelialization from the wound margins, robust granulation tissue with high cellularity (FIG. 5), and the presence of large numbers of newly formed blood vessels. By contrast, control wounds persisted in a severely impaired state without granulation tissue and blood vessel formation.

The expression of human VEGF and IL-8 mRNA transcripts at Day 10 post-wounding suggested that the applied human keratinocytes were viable in murine wound tissue. Importantly, the data indicate that tissue regeneration was not dependent on the diabetic state of mice, as the BAWD-driven marked improvement occurred in the presence of a severely diabetic phenotype. In summary, this study of a novel treatment modality for diabetic ulcers suggests that BAWD stimulates wound repair in the presence of a severely diabetic phenotype despite the underlying pathophysiological processes.

### LITERATURE

Beele H; Naeyaert J M; Goeteyn M; De Mil M; Kint A (1991): Repeated cultured epidermal allografts in the treatment of chronic leg ulcers of various origins. Dermatologica, 183 (1) 31-5.
De Luca M; Albanese E; Cancedda R; Viacava A; Faggioni A; Zambruno G; Giannetti A (1992): Treatment of leg ulcers with cryopreserved allogeneic cultured epithelium. A multicenter study. Archives of Dermatology, 128 (5) 633-8.

Härle-Bachor C; Boukamp P (1993) Telomerase activity in the regenerative basal layer of the epidermis in human skin and in immortal and carcinoma-derived skin keratinocytes. PNAS 93; 6476-6481
Falanga V et al. (1998): Rapid Healing of venous ulcers and lack of clinical rejection with an allogeneic cultured human skin equivalent. Archives of Dermatology, 134, 293-300
Harris P A; Di Franncesco F; Barisoni D; Leigh I M; Navasaria (1999): Use of hyaluronic acid and cultured autologous keratinocytes and fibroblasts in extensive burns. Lancet, 353, 35-36
Kswai K, Ikarashi Y et al. (1993): Rejection of cultured keratinocyte allografts in persensitized mice. Transplantation 56: 265-269
Lam P K; Chan E S Y; Edward W H et al. (1999): Development and evaluation of a new composite Laserskin graft. J. of Trauma: Injury, Infection and Critical Care, 47; 918-922
Lang E; Schafer B M; Eickhoff U; Hohl H P; Kramer, M D; Maier-Reif K (1996): Rapid Normalization of epidermal integrin expression after allografting of human keratinocytes. Journal of Investigative Dermatology, 107, 423-427
Leigh I M; Naysaria H; Purkis P E; McKay I (1991): Clinical practice and biological effects of keratinocyte grafting. Annals of the Academy of Medicine, 20 (4)
Lindgren C; Marcusson J A; Toftgard R (1998): Treatment of venous leg ulcers with cryopreserved cultured allogeneic keratinocytes: a prospective open controlled study. British Journal of Dermatology, 139 (2) 271-5.

Maier K (1993): Transplantation von in vitro Epidermis—Chancen and Risiken. Quintessenz 3:289-304
Phillips T J; Gilchrest B A (1989): Cultured allogenic keratinocyte grafts in the management of wound healing: prognostic factors. Journal of Dermatologic Surgery and Oncology, 15 (11)
Reinwald, J G and Green (1975): Serial cultivation of strains of human epidermal keratinocytes: the formation of keratinizing colonies from single cells. Cell 6, 331-344.
Schönfeld M; Moll I; Maier K; Jung E G (1993): Keratinozyten aus der Zellkultur zur Therapie von Hautdefekten. Hautarzt, 44:281-289
Schopp V M; Mirancea N; Fusenig N E. (1999): Epidermal Organization and Differentiation of HaCaT Keratinocytes in Organotypic Coculture with Human dermal Fibroblasts. J. Invest. Dermatology 112. 343-353
Tanczos E; Horch R E; Bannasch H; Andree C; Walgenbach K J; Voigt M; Stark G B (1999): Keratinozytentransplantation and Tissue Engineering. Neue Ansatze in der Behandlung chronischer Wunden. Zentralbl Chir 124 Suppl 1, 81-86
Teepe R G C; Roseeuw D I; Hermans J.; Koebrugge E J; Altena T; De Coninck A; Ponec M; Jan Vermeer B (1993): Randomized trial comparing cryopreserved cultured epidermal allografts with hydrocolloid dressings in healing chronic venous ulcers. Journal of the American Academy of Dermatology, 29/6 (982-988).
Wagner G; Horch R; Debus M; Tanczos E; Jiao X J; Saied S; Stark G. B. (1997): Human keratinocytes cultured subconfluent on esterified hyaluronic acid membranes for resurface full thickness nude mice wounds. European Journal of Cell Biology, 74, No. 47, pp. 61.

What is claimed is:

1. A method for treating wounds, the method comprising administering keratinocytes in combination with a DPP-4 inhibitor to a patient in need thereof, wherein the keratinocytes are not immortalised and may be doubled at least 150 times by in vitro cell culture methods.

2. The method according to claim 1, wherein the keratinocytes are isolated from the epidermal parts of a foreskin.

3. The method according to claim 1, characterised in that the keratinocytes are cells from the culture KC-BI-1 (DSM ACC 2514), or keratinocytes derived therefrom.

4. The method according to claim 1, wherein said keratinocytes
a. cannot be replicated in the absence of foetal calf serum and/or
b. in the absence of feeder cells and/or
c. in the absence of Epidermal Growth Factor (EGF).

5. The method according to claim 1, wherein the said keratinocytes have little or no telomerase activity in comparison to immortalised keratinocytes.

6. The method according to claim 1, characterised in that the said keratinocytes can be replicated at least 200 times by in vitro cell culture methods.

7. The method according to claim 1, characterised in that the said keratinocytes can be replicated at least 250 times by in vitro cell culture methods.

8. The method according to claim 1, characterised in that the said keratinocytes can be replicated at least 300 times by in vitro cell culture methods.

9. A pharmaceutical combination comprising a DPP-4 inhibitor and a carrier comprising keratinocytes, wherein
a. the carrier is partially colonised with keratinocytes; or
b. the carrier is completely colonised with keratinocytes; and
wherein the keratinocytes are not immortalised and may be doubled at least 150 times by in vitro cell culture methods.

10. The pharmaceutical combination according to claim 9, characterised in that the carrier is a biocompatible carrier material.

11. The pharmaceutical combination according to claim 10, characterised in that the carrier material is a hydrophobic or hydrophilic biodegradable membrane.

12. The pharmaceutical combination according to claim 10, characterised in that the carrier is a perforated polymer film of esterified hyaluronic acid of defined geometry, wherein the polymer film has a thickness of 10 to 500 μm and is perforated with holes measuring between 10 and 1000 μm, the holes having a defined, constant size and forming an ordered row, in which they are separated from one another by a constant spacing of 50 to 1000 μm.

13. The pharmaceutical combination according to claim 10, characterised in that the carrier material is selected from the group consisted of polyester, polycarbonates, polyanhydrides, polyorthoesters, polydepsipeptides, polyetheresters, polyamino acids or polyphosphazenes, wherein the said polymers are perforated or not perforated.

14. A process for cryopreserving keratinocytes that are not immortalised and may be doubled at least 150 times by in vitro cell culture methods, wherein the keratinocytes are cryopreserved at a temperature of −20° C. to −196° C.

15. Keratinocytes prepared by the process according to claim 14.

16. A method of using the combination according to claim 10 for treating wounds.

17. The method according to claim 16, wherein the wounds are burns and/or ulcers.

18. The method according to claim 16, wherein the wounds are second degree burns.

19. The method according to claim 16, wherein the wounds are chronic, difficult to heal, lower leg ulcers of the type Ulcus cruris, preferably Ulcus cruris venosum.

20. The method according to claim 16, wherein the wounds are ulcers caused by diabetes.

21. The method according to claim 16, wherein the wounds are decubital ulcers.

22. A method of using claim keratinocytes as a supplement to or in conjunction with one or more other substances that has a beneficial effect on wound healing, wherein the keratinocytes are not immortalised and may be doubled at least 150 times by in vitro cell culture methods, and wherein the keratinocytes are cryopreserved at a temperature of −20° C. to −196° C.

23. The method according to claim 22, wherein the other substance is a hydrocolloid dressing.

24. The method according to claim 22, wherein the other substance is an antimicrobial substance.

25. The method according to claim 1, wherein the DPP-4 inhibitor is linagliptin.

26. The method according to claim 25, wherein the linagliptin is administered or applied topically and the keratinocytes are administered or applied topically.

27. The method according to claim 25, wherein linagliptin is administered orally and the keratinocytes are administered or applied topically.

28. The pharmaceutical combination according to claim 9, wherein the keratinocytes and the DPP-4 inhibitor, are present in the same topical application form.

29. The pharmaceutical combination according to claim 28, wherein the DPP-IV inhibitor is linagliptin.
